(19) United States
(12) Patent Application Publication
(10) Pub. No.: US 2010/0087433 A1
(43) Pub. Date: Apr. 8, 2010

(54) Methods of inhibiting tryptophan hydroxylase

(76) Inventors: Catherine Bomont, Bridgewater, NJ (US); Arokiasamy Devasagayaraj, Plainsboro, NJ (US); Haihong Jin, Manalapan, NJ (US); Brett Marinelli, Hamilton, NJ (US); Lakshama Samala, Princeton, NJ (US); Zhi-Cai Shi, Monmouth Junction, NJ (US); Ashok Tunoori, Princeton, NJ (US); Ying Wang, Plainsboro, NJ (US)

Correspondence Address: LEXICON PHARMACEUTICALS, INC., 8800 TECHNOLOGY FOREST PLACE, THE WOODLANDS, TX, 77381-1160, US

(21) Appl. No.: 12/571,912

(22) Filed: Oct. 1, 2009

Related U.S. Application Data
(60) Provisional application No. 61/102,391, filed on Oct. 3, 2008.

(51) Int. Cl. A61K 31/5377; A61K 31/4418; A61K 31/444; A61P 25/00

(52) U.S. Cl. 514/235.5; 514/352; 514/334

(57) ABSTRACT

Compounds, compositions and methods of treating serotonin-mediated diseases and disorders are disclosed.

This application claims priority to U.S. provisional application No. 61/102,391, filed Oct. 3, 2008, the entirety of which is incorporated herein by reference.

### 1. FIELD OF THE INVENTION

This invention relates to multicyclic compounds, compositions comprising them, and their use in the treatment, prevention and management of diseases and disorders.

### 2. BACKGROUND

The neurotransmitter serotonin [5-hydroxytryptamine (5-HT)] is involved in multiple central nervous facets of mood control and in regulating sleep, anxiety, alcoholism, drug abuse, food intake, and sexual behavior. In peripheral tissues, serotonin is reportedly implicated in the regulation of vascular tone, gut motility, primary hemostasis, and cell-mediated immune responses. Walther, D. J., et al., Science 299:76 (2003).

The enzyme tryptophan hydroxylase (TPH) catalyzes the rate limiting step of the biosynthesis of serotonin. Two isoforms of TPH have been reported: TPH1, which is expressed in the periphery, primarily in the gastrointestinal (GI) tract; and TPH2, which is expressed in the serotonergic neurons. Id. The isoform TPH1 is encoded by the tph1 gene; TPH2 is encoded by the tph2 gene. Id.

Mice genetically deficient for the tph1 gene (“knockout mice”) have been reported. In one case, the mice reportedly expressed normal amounts of serotonin in classical serotonergic brain regions, but largely lacked serotonin in the periphery. Id. In another, the knockout mice exhibited abnormal cardiac activity, which was attributed to a lack of peripheral serotonin. Cote, F., et al., PNAS 100(23):13525-13530 (2003).

Compounds that inhibit TPH and methods of their use have been disclosed. See, e.g., U.S. patent application Ser. Nos. 11/638,677 and 11/954,000. Because serotonin is involved in so many biochemical processes, a need exists for additional compounds and methods of treating diseases and disorders mediated by peripheral serotonin.

### 3. SUMMARY OF THE INVENTION

This invention is directed, in part, to compounds of the formula:

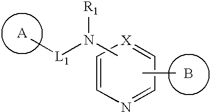

the substituents of which are defined herein. The invention also encompasses compounds of the formula:

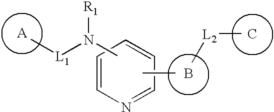

the substituents of which are defined herein. Also encompassed are compounds of the formula:

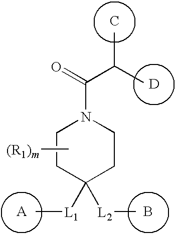

the substituents of which are defined herein.

Particular compounds of the invention (i.e., compounds described herein) inhibit TPH (e.g., TPH1) activity.

This invention is also directed to pharmaceutical compositions and to methods of treating, preventing and managing a variety of diseases and disorders.

### 4. DETAILED DESCRIPTION

This invention is based on the discovery of compounds that inhibit TPH (e.g., TPH1), and which may be used to treat, manage or prevent diseases and disorders mediated by peripheral serotonin.

### 4.1. Definitions

Unless otherwise indicated, the term “alkenyl” means a straight chain, branched and/or cyclic hydrocarbon having from 2 to 20 (e.g., 2 to 10 or 2 to 6) carbon atoms, and including at least one carbon-carbon double bond. Representative alkenyl moieties include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl and 3-decenyl.

Unless otherwise indicated, the term “alkyl” means a straight chain, branched and/or cyclic (“cycloalkyl”) hydrocarbon having from 1 to 20 (e.g., 1 to 10 or 1 to 4) carbon atoms. Alkyl moieties having from 1 to 4 carbons are referred to as “lower alkyl.” Examples of alkyl groups include methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl and dodecyl. Cycloalkyl moieties may be monocyclic or multicyclic, and examples include cyclopropyl, cyclobutyl, cyclopentyl, cyclohexyl, and adamantyl. Additional examples of alkyl moieties have linear, branched and/or cyclic portions (e.g., 1-ethyl-4-methyl-cyclohexyl). The term “alkyl” includes saturated hydrocarbons as well as alkenyl and alkynyl moieties.

Unless otherwise indicated, the term “alkoxy” means an —O-alkyl group. Examples of alkoxy groups include —OCH₃, —OCH₂CH₃, —O(CH₂)₂CH₃, —O(CH₂)₃CH₃, —O(CH₂)₄CH₃, —O(cyclopenyl) and —O(CH₂)₅CH₃.

Unless otherwise indicated, the term “alkylaryl” or “alkyl-aryl” means an alkyl moiety bound to an aryl moiety.

Unless otherwise indicated, the term “alkylheteroaryl” or “alkyl-heteroaryl” means an alkyl moiety bound to a heteroaryl moiety.

Unless otherwise indicated, the term “alkylheterocycle” or “alkyl-heterocycle” means an alkyl moiety bound to a heterocycle moiety.

Unless otherwise indicated, the term “alkynyl” means a straight chain, branched or cyclic hydrocarbon having from 2 to 20 (e.g., 2 to 20 or 2 to 6) carbon atoms, and including at least one carbon-carbon triple bond. Representative alkynyl moieties include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl and 9-decynyl.

Unless otherwise indicated, the term “aryl” means an aromatic ring or an aromatic or partially aromatic ring system composed of carbon and hydrogen atoms. An aryl moiety may comprise multiple rings bound or fused together. Examples of aryl moieties include anthracenyl, azulenyl, biphenyl, fluorenyl, indan, indenyl, naphthyl, phenanthrenyl, phenyl, 1,2,3,4-tetrahydro-naphthalene, and tolyl.

Unless otherwise indicated, the term “arylalkyl” or “aryl-alkyl” means an aryl moiety bound to an alkyl moiety.

Unless otherwise indicated, the terms “biohydrolyzable amide,” “biohydrolyzable ester,” “biohydrolyzable carbamate,” “biohydrolyzable carbonate,” “biohydrolyzable ureido” and “biohydrolyzable phosphate” mean an amide, ester, carbamate, carbonate, ureido, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include lower alkyl esters, alkoxyacyloxy esters, alkyl acylamino alkyl esters, and choline esters. Examples of biohydrolyzable amides include lower alkyl amides, α-amino acid amides, alkoxyacyl amides, and alkylaminoalkyl-carbonyl amides. Examples of biohydrolyzable carbamates include lower alkylamines, substituted ethylenediamines, aminoacids, hydroxyalkylamines, heterocyclic and heteroaromatic amines, and polyether amines.

Unless otherwise indicated, the phrases “disease or disorder mediated by peripheral serotonin” and “disease and disorder mediated by peripheral serotonin” mean a disease and/or disorder having one or more symptoms, the severity of which are affected by peripheral serotonin levels.

Unless otherwise indicated, the terms “halogen” and “halo” encompass fluorine, chlorine, bromine, and iodine.

Unless otherwise indicated, the term “heteroalkyl” refers to an alkyl moiety (e.g., linear, branched or cyclic) in which at least one of its carbon atoms has been replaced with a heteroatom (e.g., N, O or S).

Unless otherwise indicated, the term “heteroaryl” means an aryl moiety wherein at least one of its carbon atoms has been replaced with a heteroatom (e.g., N, O or S). Examples include acridinyl, benzimidazolyl, benzofuranyl, benzoisothiazolyl, benzoisoxazolyl, benzoquinazolinyl, benzothiazolyl, benzoxazolyl, furyl, imidazolyl, indolyl, isothiazolyl, isoxazolyl, oxadiazolyl, oxazolyl, phthalazinyl, pyrazinyl, pyrazolyl, pyridazinyl, pyridyl, pyrimidinyl, pyrimidyl, pyrrolyl, quinazolinyl, quinolinyl, tetrazolyl, thiazolyl, and triazinyl.

Unless otherwise indicated, the term “heteroarylalkyl” or “heteroaryl-alkyl” means a heteroaryl moiety bound to an alkyl moiety.

Unless otherwise indicated, the term “heterocycle” refers to an aromatic, partially aromatic or non-aromatic monocyclic or polycyclic ring or ring system comprised of carbon, hydrogen and at least one heteroatom (e.g., N, O or S). A heterocycle may comprise multiple (i.e., two or more) rings fused or bound together. Heterocycles include heteroaryls. Particular heterocycles are 5- to 13-membered heterocycles containing 1 to 4 heteroatoms selected from nitrogen, oxygen, and sulphur. Others are 5- to 10-membered heterocycles containing 1 to 4 heteroatoms selected from nitrogen, oxygen, and sulphur. Examples of heterocycles include benzo[1,3]dioxolyl, 2,3-dihydro-benzo[1,4]dioxinyl, cinnolinyl, furanyl, hydantoinyl, morpholinyl, oxetanyl, oxiranyl, piperazinyl, piperidinyl, pyrrolidinonyl, pyrrolidinyl, tetrahydrofuranyl, tetrahydropyranyl, tetrahydropyridinyl, tetrahydropyrimidinyl, tetrahydrothiophenyl, tetrahydrothiopyranyl and valerolactamyl.

Unless otherwise indicated, the term “heterocyclealkyl” or “heterocycle-alkyl” refers to a heterocycle moiety bound to an alkyl moiety.

Unless otherwise indicated, the term “heterocycloalkyl” refers to a non-aromatic heterocycle.

Unless otherwise indicated, the term “heterocycloalkylalkyl” or “heterocycloalkyl-alkyl” refers to a heterocycloalkyl moiety bound to an alkyl moeity.

Unless otherwise indicated, the terms “manage,” “managing” and “management” encompass preventing the recurrence of the specified disease or disorder, or of one or more of its symptoms, in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

Unless otherwise indicated, the term “pharmaceutically acceptable salts” refers to salts prepared from pharmaceutically acceptable non-toxic acids or bases including inorganic acids and bases and organic acids and bases. Suitable pharmaceutically acceptable base addition salts include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art. See, e.g., Remington's Pharmaceutical Sciences, 18^{th} ed. (Mack Publishing, Easton Pa.: 1990) and Remington: The Science and Practice of Pharmacy, 19^{th} ed. (Mack Publishing, Easton Pa.: 1995).

Unless otherwise indicated, the terms “prevent,” “preventing” and “prevention” contemplate an action that occurs before a patient begins to suffer from the specified disease or disorder, which inhibits or reduces the severity of the disease or disorder or of one or more of its symptoms. The terms encompass prophylaxis.

Unless otherwise indicated, the term “prodrug” encompasses pharmaceutically acceptable esters, carbonates, thiocarbonates, N-acyl derivatives, N-acyloxyalkyl derivatives, quaternary derivatives of tertiary amines, N-Mannich bases, Schiff bases, aminoacid conjugates, phosphate esters, metal salts and sulfonate esters of compounds disclosed herein. Examples of prodrugs include compounds that comprise a biohydrolyzable moiety (e.g., a biohydrolyzable amide, biohydrolyzable carbamate, biohydrolyzable carbonate, biohydrolyzable ester, biohydrolyzable phosphate, or biohydrolyzable ureide analog). Prodrugs of compounds disclosed herein are readily envisioned and prepared by those of ordinary skill in the art. See, e.g., Design of Prodrugs, Bundgaard, A. Ed., Elseview, 1985; Bundgaard, hours., “Design and Application of Prodrugs,” A Textbook of Drug Design and Development, Krosgaard-Larsen and hours. Bundgaard, Ed., 1991, Chapter 5, p. 113-191; and Bundgaard, hours., Advanced Drug Delivery Review, 1992, 8, 1-38.

Unless otherwise indicated, a “prophylactically effective amount” of a compound is an amount sufficient to prevent a disease or condition, or one or more symptoms associated with the disease or condition, or prevent its recurrence. A prophylactically effective amount of a compound is an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

Unless otherwise indicated, the term “protecting group” or “protective group,” when used to refer to part of a molecule subjected to a chemical reaction, means a chemical moiety that is not reactive under the conditions of that chemical reaction, and which may be removed to provide a moiety that is reactive under those conditions. Protecting groups are well known in the art. See, e.g., Greene, T. W. and Wuts, P.G.M., Protective Groups in Organic Synthesis (3^{rd} ed., John Wiley & Sons: 1999); Larock, R. C., Comprehensive Organic Transformations (2^{nd} ed., John Wiley & Sons: 1999). Some examples include benzyl, diphenylmethyl, trityl, Cbz, Boc, Fmoc, methoxycarbonyl, ethoxycarbonyl, and pthalimido.

Unless otherwise indicated, the term “stereomerically enriched composition of” a compound refers to a mixture of the named compound and its stereoisomer(s) that contains more of the named compound than its stereoisomer(s). For example, a stereoisomerically enriched composition of (S)-butan-2-ol encompasses mixtures of (S)-butan-2-ol and (R)-butan-2-ol in ratios of, e.g., about 60/40, 70/30, 80/20, 90/10, 95/5, and 98/2.

Unless otherwise indicated, the term “stereoisomeric mixture” encompasses racemic mixtures as well as stereomerically enriched mixtures (e.g., R/S=30/70, 35/65, 40/60, 45/55, 55/45, 60/40, 65/35 and 70/30).

Unless otherwise indicated, the term “stereomerically pure” means a composition that comprises one stereoisomer of a compound and is substantially free of other stereoisomers of that compound. For example, a stereomerically pure composition of a compound having one stereocenter will be substantially free of the opposite stereoisomer of the compound. A stereomerically pure composition of a compound having two stereocenters will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound.

Unless otherwise indicated, the term “substituted,” when used to describe a chemical structure or moiety, refers to a derivative of that structure or moiety wherein one or more of its hydrogen atoms is substituted with a chemical moiety or functional group such as, but not limited to, alcohol, aldehylde, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl -or -alkylNHC(O)alkyl), amidinyl (—C(NH)NH-alkyl or —C(NR)NH₂), amine (primary, secondary and tertiary such as alkylamino, arylamino, arylalkylamino), aroyl, aryl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carbonyl, carboxyl, carboxylic acid, carboxylic acid anhydride, carboxylic acid chloride, cyano, ester, epoxide, ether (e.g., methoxy, ethoxy), guanidino, halo, haloalkyl (e.g., —CCl₃, —CF₃, —C(CF₃)₃), heteroalkyl, hemiacetal, imine (primary and secondary), isocyanate, isothiocyanate, ketone, nitrile, nitro, oxo, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) and urea (—NHCONH-alkyl-). Particular substituents are alkyl, alkyl-carbamyl, alkoxy, amino, halo, hydroxyl, nitro, sulfonyl (e.g., methylsulfonyl, tosyl), and thiol.

Unless otherwise indicated, a “therapeutically effective amount” of a compound is an amount sufficient to provide a therapeutic benefit in the treatment or management of a disease or condition, or to delay or minimize one or more symptoms associated with the disease or condition. A therapeutically effective amount of a compound is an amount of therapeutic agent, alone or in combination with other therapies, which provides a therapeutic benefit in the treatment or management of the disease or condition. The term “therapeutically effective amount” can encompass an amount that improves overall therapy, reduces or avoids symptoms or causes of a disease or condition, or enhances the therapeutic efficacy of another therapeutic agent.

Unless otherwise indicated, the term “TPH inhibitor” refers to a compound that has a TPH1_IC₅₀ or TPH2_IC₅₀ that is less than about 10 μM. Particular TPH inhibitors have a TPH1_IC₅₀ that is less than about 5, 1, 0.5, 0.1 or 0.05 μM.

Unless otherwise indicated, the term “TPH1_IC₅₀” is the IC₅₀ of a compound for TPH1 as determined using the in vitro inhibition assay described in the Examples, below.

Unless otherwise indicated, the term “TPH2_IC₅₀” is the IC₅₀ of a compound for TPH2 as determined using the in vitro inhibition assay described in the Examples, below.

Unless otherwise indicated, the terms “treat,” “treating” and “treatment” contemplate an action that occurs while a patient is suffering from the specified disease or disorder, which reduces the severity of the disease or disorder, or one or more of its symptoms, or retards or slows the progression of the disease or disorder.

Unless otherwise indicated, the term “include” has the same meaning as “include” and the term “includes” has the same meaning as “includes, but is not limited to.” Similarly, the term “such as” has the same meaning as the term “such as, but not limited to.”

Unless otherwise indicated, one or more adjectives immediately preceding a series of nouns is to be construed as applying to each of the nouns. For example, the phrase “optionally substituted alky, aryl, or heteroaryl” has the same meaning as “optionally substituted alky, optionally substituted aryl, or optionally substituted heteroaryl.”

It should be noted that a chemical moiety that forms part of a larger compound may be described herein using a name commonly accorded it when it exists as a single molecule or a name commonly accorded its radical. For example, the terms “pyridine” and “pyridyl” are accorded the same meaning when used to describe a moiety attached to other chemical moieties. Thus, the two phrases “XOH, wherein X is pyridyl” and “XOH, wherein X is pyridine” are accorded the same meaning, and encompass the compounds pyridin-2-ol, pyridin-3-ol and pyridin-4-ol.

It should also be noted that if the stereochemistry of a structure or a portion of a structure is not indicated with, for example, bold or dashed lines, the structure or the portion of the structure is to be interpreted as encompassing all stereoisomers of it. Similarly, names of compounds having one or more chiral centers that do not specify the stereochemistry of those centers encompass pure stereoisomers and mixtures thereof. Moreover, any atom shown in a drawing with unsatisfied valences is assumed to be attached to enough hydrogen atoms to satisfy the valences. In addition, chemical bonds depicted with one solid line parallel to one dashed line encompass both single and double (e.g., aromatic) bonds, if valences permit.

### 4.2. Compounds

This invention encompasses, inter alia, compounds of Formula I:

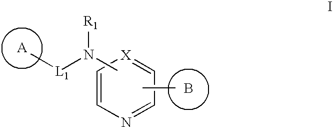

and pharmaceutically acceptable salts thereof, wherein: X is C or N; A is optionally substituted aryl or heteroaryl; B is optionally substituted aryl or heteroaryl; L₁ is —(CR₂)ₘ—; R₁ is hydrogen or optionally substituted alkyl; each R₂ is independently hydrogen or optionally substituted alkyl; and m is 0 or 1.

Particular compounds are of the formula:

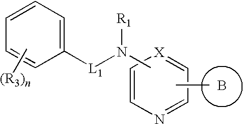

wherein: each R₃ is independently optionally substituted alkyl, heteroalkyl, aryl, heterocycle, alkylaryl, heteroalkyl-aryl, alkyl-heterocycle, or heteroalkyl-heterocycle; and n is 0-4.

With respect to the various formulae shown above and elsewhere herein, particular compounds are such that R₁ is hydrogen. In particular compounds, R₂ is hydrogen. In some compounds, at least one R₃ is alkoxy. In some, m is 0; in others, m is 1.

Particular compounds are of the formula:

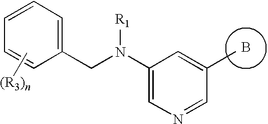

Some are of the formula:

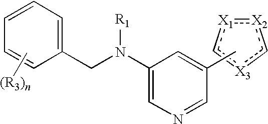

wherein: X₁ is N, NR₄, O, CHR₅, or CR₅; X₂ is N, NR₄, O, CHR₅, or CR₅; X₃ is N, NR₄, O, CHR₅, or CR₅; each R₄ is independently hydrogen or optionally substituted alkyl, heteroalkyl, aryl, heterocycle, alkylaryl, heteroalkyl-aryl, alkyl-heterocycle, or heteroalkyl-heterocycle; and each R₅ is independently hydrogen or optionally substituted alkyl, heteroalkyl, aryl, heterocycle, alkylaryl, heteroalkyl-aryl, alkyl-heterocycle, or heteroalkyl-heterocycle.

With respect to the various formulae shown above and elsewhere herein, particular compounds are such that X₁ is 0 and X₂ and X₃ are both CHR₅. In some, R₅ is hydrogen. In some compounds, X₁ is N, X₂ is NR₄, and X₃ is CHR₅. In some compounds, R₄ is optionally substituted alkyl or heteroalkyl, and R₅ is hydrogen or optionally substituted alkyl.

Particular compounds are of the formula:

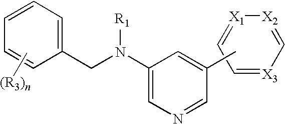

wherein: X₁ is N or CR₄; X₂ is N or CR₄; X₃ is N or CR₄; and each R₄ is independently hydrogen or optionally substituted alkyl, heteroalkyl, aryl, heterocycle, alkylaryl, heteroalkyl-aryl, alkyl-heterocycle, or heteroalkyl-heterocycle.

Particular compounds are of the formula:

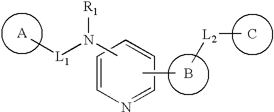

wherein: A is optionally substituted aryl or heteroaryl; B is optionally substituted aryl or heteroaryl; C is optionally substituted aryl or heteroaryl; L₁ is —(CR₂)ₘ—; L₂ is —(CR₂)ₘ—; R₁ is hydrogen or optionally substituted alkyl; each R₂ is independently hydrogen or optionally substituted alkyl; and each m is independently 0 or 1. Some are of the formula:

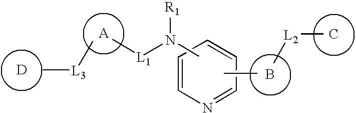

wherein: D is optionally substituted aryl or heteroaryl; L₃ is —(CR₂)ₘ— or —O—; and each m is independently 0 or 1.

One embodiment of the invention encompasses compounds of Formula II:

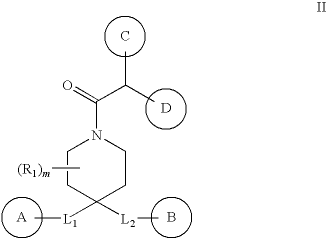

and pharmaceutically acceptable salt thereof, wherein: A is optionally substituted aryl or heteroaryl; B is optionally substituted aryl or heteroaryl; C is optionally substituted aryl or heteroaryl; D is optionally substituted aryl or heteroaryl; each R₁ is independently halo, hydroxyl, or lower alkyl; L₁ is a bond or —(CH₂)ₙ—; L₂ is a bond or —(CH₂)—; m is 0-4; and each n is independently 0-2.

With respect to the various formulae shown above and elsewhere herein, particular compounds are such that A is optionally substituted imidazole. In some, B is optionally substituted phenyl. In some, C is optionally substituted phenyl. In some, D is optionally substituted phenyl.

Particular compounds are of the formula:

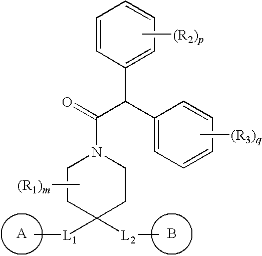

wherein: each R₂ is independently halo, hydroxyl, or lower alkyl; each R₃ is independently halo, hydroxyl, or lower alkyl; p is 0-5; and q is 0-5.

Particular compounds of the invention are TPH inhibitors.

This invention encompasses stereomerically pure compounds and stereomerically enriched compositions of them. Stereoisomers may be asymmetrically synthesized or resolved using standard techniques such as chiral columns, chiral resolving agents, or enzymatic resolution. See, e.g., Jacques, J., et al., Enantiomers, Racemates and Resolutions (Wiley Interscience, New York, 1981); Wilen, S. hours., et al., Tetrahedron 33:2725 (1977); Eliel, E. L., Stereochemistry of Carbon Compounds (McGraw Hill, N.Y., 1962); and Wilen, S. hours., Tables of Resolving Agents and Optical Resolutions, p. 268 (E. L. Eliel, Ed., Univ. of Notre Dame Press, Notre Dame, Ind., 1972).

### 4.3. Synthesis of Compounds

Compounds of the invention can be prepared by methods known in the art and by methods described herein. For example, compounds of formula I can be prepared according to the approach shown in Scheme 1, below:

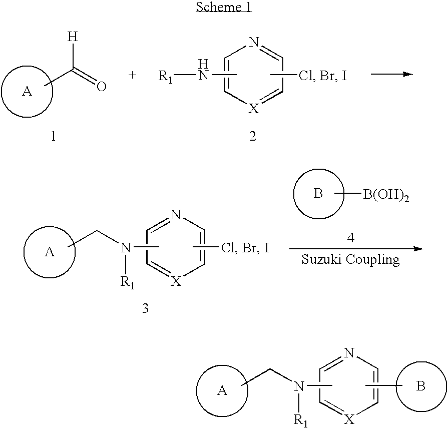

In this approach, aldehyde compound 1 and amine substituted heterocyclic halide 2 are reacted under typical reductive amination condition to give compound 3. Suitable solvents include dichloromethane, dichloroethane, methanol, and trimethyl orthoformate. Suitable reducing agents include sodium cyano borohydride, sodium triacetoxy borohydride, and sodium borohydride, and suitable acid catalysts include acetic acid and trifluoroacetic acid. Compound 3 is then coupled with the desired boronic acid 4 under Suzuki coupling conditions to afford the compound of Formula I. Both conventional heating and microwave irradiation can be used for the coupling reaction. Suitable catalysts for this reaction include Pd(PPh₃)₂Cl₂, PdCl₂, Pd(dppf)₂, Pd₂(dba)₃, Pd(OAc)₂, and Pd-EnCat, Pd(PPh₃)₄. Suitable bases include Na₂CO₃, NaHCO₃, K₂CO₃, KOAc, and Cs₂CO₃, KF, and suitable solvents include DMF, DMSO, ethanol, MeOH, 1,4-dioxane, THF, CH₃CN, and water.

Compounds of Formula I can also be prepared by the approach shown below in Scheme 2, using reaction conditions similar to those described above:

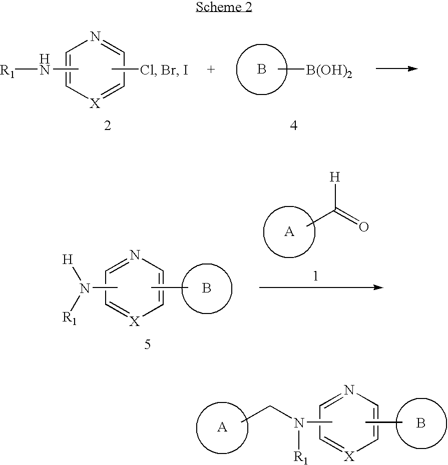

Compounds of Formula II can generally be prepared using the approach shown below in Scheme 3:

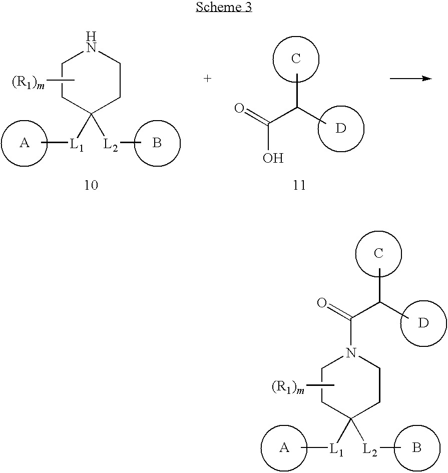

In this approach, a substituted piperidine 10 is coupled with a carboxylic acid 11 under amide bond formation conditions to afford a compound of Formula II. Typical coupling reagents include N,N′-dicylohexylcarbodiimide (DCC)/1-hydroxyl benzotriazole (HOBt), N,N′-diisopropylcarbodiimide (DIC)/HOBt, polymer bound-DCC/HOBt, bromotripyrrolidinophosphonium hexafluorophosphate (PyBroP)/Hunig's base, PyBOP/Hunig's base, and O-(7-Azabenotriazol-1-yl)-N,N,N′,N′-tetramethyluronium hexafluorophosphate (HATU).

Using methods known in the art, the synthetic approaches described herein are readily modified to obtain a wide range of compounds. For example, chiral chromatography and other techniques known in the art may be used to separate stereoisomers of the final product. See, e.g., Jacques, J., et al., Enantiomers, Racemates and Resolutions (Wiley Interscience, New York, 1981); Wilen, S. hours., et al., Tetrahedron 33:2725 (1977); Eliel, E. L., Stereochemistry of Carbon Compounds (McGraw Hill, N.Y., 1962); and Wilen, S. hours., Tables of Resolving Agents and Optical Resolutions, p. 268 (E. L. Eliel, Ed., Univ. of Notre Dame Press, Notre Dame, Ind., 1972). In addition, syntheses may utilize chiral starting materials to yield stereomerically enriched or pure products.

### 4.4. Methods of Use

This invention encompasses a method of inhibiting TPH, which comprises contacting TPH with a compound of the invention (i.e., a compound disclosed herein). In a particular method, the TPH is TPH1. In another, the TPH is TPH2. In a particular method, the inhibition is in vitro. In another, the inhibition is in vivo.

This invention encompasses methods of treating, preventing and managing diseases and disorders mediated by peripheral serotonin, which comprise administering to a patient in need of such treatment, prevention or management a therapeutically or prophylactically effective amount of a compound of the invention.

Particular diseases and disorders are associated with the gastrointestinal (GI) tract. Examples of specific diseases and disorders include anxiety, Bile Acid Diarrhea, carcinoid syndrome, celiac disease, Crohn's disease, depression, diabetes, diarrhea and/or abdominal pain associated with medullary carcinoma of the thyroid, enterotoxin-induced secretory diarrhea, functional abdominal pain, functional dyspepsia, idiopathic constipation, iatrogenic causes of constipation and/or diarrhea, idiopathic diarrhea (e.g., idiopathic secretory diarrhea), irritable bowel syndrome (IBS), lactose intolerance, MEN types I and II, Ogilvie's syndrome, Pancreatic Cholera Syndrome, pancreatic insufficiency, pheochromacytoma, scleroderma, somatization disorder, traveler's diarrhea, ulcerative colitis, and Zollinger-Ellison Syndrome. Others include functional anorectal disorders, functional bloating, and functional gallbladder and sphincter of Oddi disorders.

Others are cardiovascular and pulmonary diseases and disorders, such as acute and chronic hypertension, chronic obstructive pulmonary disease (COPD), pulmonary embolism (e.g., bronchoconstriction and pulmonary hypertension following pulmonary embolism), pulmonary hypertension (e.g., pulmonary hypertension associated with portal hypertension), and radiation pneumonitis (including that giving rise to or contributing to pulmonary hypertension).

Still others include abdominal migraine, adult respiratory distress syndrome (ARDS), carcinoid crisis, CREST syndrome (calcinosis, Raynaud's phenomenon, esophageal dysfunction, sclerodactyl), telangiectasia), Gilbert's syndrome, nausea, serotonin syndrome, and subarachnoid hemorrhage.

### 4.5. Pharmaceutical Compositions

This invention encompasses pharmaceutical compositions comprising one or more compounds of the invention. Certain pharmaceutical compositions are single unit dosage forms suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The formulation should suit the mode of administration. For example, the oral administration of a compound susceptible to degradation in the stomach may be achieved using an enteric coating. Similarly, a formulation may contain ingredients that facilitate delivery of the active ingredient(s) to the site of action. For example, compounds may be administered in liposomal formulations in order to protect them from degradative enzymes, facilitate transport in circulatory system, and effect their delivery across cell membranes.

Similarly, poorly soluble compounds may be incorporated into liquid dosage forms (and dosage forms suitable for reconstitution) with the aid of solubilizing agents, emulsifiers and surfactants such as, but not limited to, cyclodextrins (e.g., α-cyclodextrin, β-cyclodextrin, Captisol®, and Encapsin™ (see, e.g., Davis and Brewster, Nat. Rev. Drug Disc. 3:1023-1034 (2004)), Labrasol®, Labrafil®, Labrafac®, cremafor, and non-aqueous solvents, such as, but not limited to, ethyl alcohol, isopropyl alcohol, ethyl carbonate, ethyl acetate, benzyl alcohol, benzyl benzoate, propylene glycol, 1,3-butylene glycol, dimethyl formamide, dimethyl sulfoxide (DMSO), biocompatible oils (e.g., cottonseed, groundnut, corn, germ, olive, castor, and sesame oils), glycerol, tetrahydrofurfuryl alcohol, polyethylene glycols, fatty acid esters of sorbitan, and mixtures thereof (e.g., DMSO:cornoil).

Poorly soluble compounds may also be incorporated into suspensions using other techniques known in the art. For example, nanoparticles of a compound may be suspended in a liquid to provide a nanosuspension (see, e.g., Rabinow, Nature Rev. Drug Disc. 3:785-796 (2004)). Nanoparticle forms of compounds described herein may be prepared by the methods described in U.S. Patent Publication Nos. 2004-0164194, 2004-0195413, 2004-0251332, 2005-0042177 A1, 2005-0031691 A1, and U.S. Pat. Nos. 5,145,684, 5,510,118, 5,518,187, 5,534,270, 5,543,133, 5,662,883, 5,665,331, 5,718,388, 5,718,919, 5,834,025, 5,862,999, 6,431,478, 6,742,734, 6,745,962, the entireties of each of which are incorporated herein by reference. In one embodiment, the nanoparticle form comprises particles having an average particle size of less than about 2000 nm, less than about 1000 nm, or less than about 500 nm.

The composition, shape, and type of a dosage form will typically vary depending with use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. How to account for such differences will be apparent to those skilled in the art. See, e.g., Remington's Pharmaceutical Sciences, 18th ed., Mack Publishing, Easton Pa. (1990).

### 4.5.1. Oral Dosage Forms

Pharmaceutical compositions of the invention suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms are prepared by combining the active ingredient(s) in an intimate admixture with at least one excipient according to conventional pharmaceutical compounding techniques. Excipients can take a wide variety of forms depending on the form of preparation desired for administration.

Because of their ease of administration, tablets and capsules represent the most advantageous oral dosage unit forms. If desired, tablets can be coated by standard aqueous or nonaqueous techniques. Such dosage forms can be prepared by conventional methods of pharmacy. In general, pharmaceutical compositions and dosage forms are prepared by uniformly and intimately admixing the active ingredients with liquid carriers, finely divided solid carriers, or both, and then shaping the product into the desired presentation if necessary. Disintegrants may be incorporated in solid dosage forms to facility rapid dissolution. Lubricants may also be incorporated to facilitate the manufacture of dosage forms (e.g., tablets).

### 4.5.2. Parenteral Dosage Forms

Parenteral dosage forms can be administered to patients by various routes including subcutaneous, intravenous (including bolus injection), intramuscular, and intraarterial. Because their administration typically bypasses patients' natural defenses against contaminants, parenteral dosage forms are specifically sterile or capable of being sterilized prior to administration to a patient. Examples of parenteral dosage forms include solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include: Water for Injection USP; aqueous vehicles such as Sodium Chloride Injection, Ringer's Injection, Dextrose Injection, Dextrose and Sodium Chloride Injection, and Lactated Ringer's Injection; water-miscible vehicles such as ethyl alcohol, polyethylene glycol, and polypropylene glycol; and non-aqueous vehicles such as corn oil, cottonseed oil, peanut oil, sesame oil, ethyl oleate, isopropyl myristate, and benzyl benzoate.

### 5. EXAMPLES

### 5.1. Synthesis of 3-(5-(3-(cyclopentyloxy)-4-methoxybenzylamino)pyridin-3-yl)benzonitrile

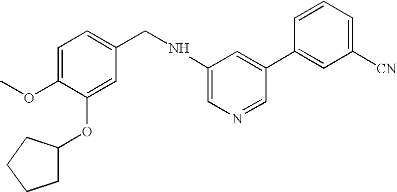

To a mixture of 3-amino-5-bromopyridine (0.64 g, 3.7 mmol) and 3-(cyclopentyloxy)-4-methoxybenzaldehyde (0.97 g, 4.4 mmol) in dicholoroethane (20 mL), was added sodium triacetoxy borohydride (1.56 g, 7.35 mmol) and acetic acid (0.3 mL). The reaction mixture was stirred at room temperature for 4 hours. Methylene chloride (100 mL) was added to reaction mixture, which was washed with 1N NaOH and brine respectively. The methylene chloride layer was separated and dried over MgSO₄. Removal of solvent gave 1.29 g of light yellow solid as crude product, which was used in the next step without further purification.

The above crude product (43.2 mg, 0.115 mmol), 3-cyanophenylboronic acid (16.8 mg, 0.115 mmol), dichlorobis(triphenylphosphine)-palladium(II) (4 mg, 0.006 mmol), CH₃CN (2 mL) and water (1.78 mL) were mixed in a vial for microwave assisted reaction. Sodium carbonate (0.22 mL, 1M aqueous) was added to the mixture, which was irradiated in Personal Chemistry microwave reactor at 150° C. for 5 minutes. The crude reaction mixture was worked up and purified by preparative HPLC to give 9.5 mg of 3-(5-(3-(cyclopentyloxy)-4-methoxybenzylamino)pyridin-3-yl)benzonitrile (Yield: 21%).

¹H NMR (300 MHz, CD₃OD) δ (ppm): 8.27 (s, 1H); 8.08 (m, 1H); 7.99 (m, 2H); 7.86 (m, 2H); 7.73 (t, 1H, J=9 Hz); 6.95 (m, 3H); 4.79 (m, 1H), 4.44 (s, 2H); 3.80 (s, 3H); 1.80 (m, 6H); 1.61 (m, 2H). HPLC: Column=Shim-pack ODS 4.6×50 mm, 5 um; Solvent A=0.1% TFA (trifluoroacetic acid) in water; Solvent B=0.1% TFA in MeOH; B % from 20 to 90% over 4 minutes at flow rate=3 ml/min, UV detector at 220 and 254 nm; RT=2.74 minutes. ESI-MS: (M+H)⁺=400.

### 5.2. Synthesis N-(3-(cyclopentyloxy)-4-methoxybenzyl)-3,3′-bipyridin-6-amine

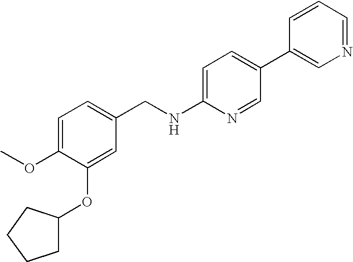

Acetic acid (900 mg, 15 mmol) was added to a solution of 3-(cyclopentyloxy)-4-methoxybenzaldehyde (1.1 g, 5 mmol), 5-iodopyridin-2-amine (1.1 g, 5 mmol) and sodium triacetoxyborohydride (1.4 g, 6.6 mmol) in 30 mL dichloroethane at room temperature. The resulting mixture was heated at 60° C. for 4 hours. The reaction mixture was quenched with water. The product was extracted with dichloromethane (3×20 ml). The organic layer was separated and dried over sodium sulfate. The organic solvent was evaporated to dryness. The crude product was purified by SiO₂ column chromatography to give 1.2 g of N-(3-(cyclopentyloxy)-4-methoxybenzyl)-5-iodopyridin-2-amine. Yield: 64%

Microwave vial (2 mL) was charged with N-(3-(cyclopentyloxy)-4-methoxybenzyl)-5-iodopyridin-2-amine (42 mg, 0.1 mmol) and pyridin-3-ylboronic acid (12 mg, 0.1 mmol). Then acetonitrile (1 mL), water (0.8 mL), aqueous sodium carbonate (0.2 ml, 1M) and dichlorobis(triphenylphosphine)-palladium(II) (5 mg, 0.007 mmol) were added into the mixture. The reaction vessel was sealed and heated at 150° C. for 5 minutes under microwave irradiation. After cooling, the reaction mixture was worked up and purified with preparative HPLC to give 8 mg of N-(3-(cyclopentyloxy)-4-methoxybenzyl)-3,3′ bipyridin-6-amine.

¹H NMR (300 MHz, CD₃Cl) δ (ppm): 9.00 (s, 1H), 8.73 (s, 1H), 8.17 (m, 2H), 8.00 (m, 1H), 7.78 (m, 1H), 6.86 (m, 5H), 4.80 (m, 1H), 4.54 (s, 2H), 3.83 (s, 3H), 1.91 (m, 6H), 1.60 (m, 2H). HPLC: column=YMC Pack ODS-AQ 3.0×50 mm, 5 um; Solvent A=0.1% TFA (trifluoroacetic acid) in water/MeOH (90/10); Solvent B=0.1% TFA in MeOH/water (90/10); B % from 0 to 100% over 5 minutes at flow rate=2.0 ml/min, RT=2.232 minutes. ESI-MS: m/z (M+H)⁺=376.

### 5.3. Synthesis N-(3-(cyclopentyloxy)-4-methoxybenzyl)-3,3′ bipyridin-5-amine

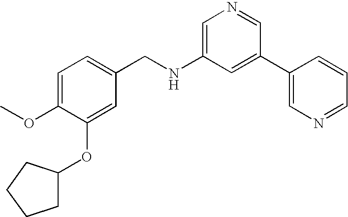

Acetic acid (414 mg, 6.9 mmol) was added to a solution of 3-(cyclopentyloxy)-4-methoxybenzaldehyde (508 mg, 2.3 mmol), 5-bromopyridin-3-amine (400 mg, 2.3 mmol) and sodium triacetoxyborohydride (0.65 g, 3.1 mmol) in 30 ml DCE at room temperature. The formed mixture was warmed up to 60° C. and stirred for 4 hours. The reaction mixture was quenched with water. The product was extracted with DCM (3×20 ml). The organic layer was separated and dried over sodium sulfate. The organic solvent was evaporated to dryness. The crude product was purified by SiO₂ column chromatography to give 350 mg of 5-bromo-N-(3-(cyclopentyloxy)-4-methoxybenzyl)pyridin-3-amine. Yield: 41%

Microwave vial (2 mL) was charged with 5-bromo-N-(3-(cyclopentyloxy)-4-methoxybenzyl)pyridin-3-amine (38 mg, 0.1 mmol) and pyridin-3-ylboronic acid (13 mg, 0.1 mmol). Then, acetonitrile (1 mL), water (0.8 mL), aqueous sodium carbonate (0.2 mL, 1M) and dichlorobis(triphenylphosphine)-palladium(II) (5 mg, 0.007 mmol) were added to the mixture. The reaction vessel was sealed and heated at 150° C. for 5 minutes under microwave irradiation. After cooling, the reaction mixture was worked up and purified by preparative HPLC to give 8 mg of N-(3-(cyclopentyloxy)-4-methoxybenzyl)-3,3′ bipyridin-5-amine.

¹H NMR (300 MHz, CD₃Cl) δ (ppm): 8.88 (s, 1H), 8.76 (s, 1H), 8.38 (m, 2H), 8.18 (s, 1H), 8.03 (m, 1H), 7.65 (m, 1H), 7.28 (s, 1H), 6.86 (m, 2H), 4.77 (m, 1H), 4.42 (s, 2H), 3.84 (s, 3H), 1.91 (m, 6H), 1.60 (m, 2H). HPLC: column=YMC Pack ODS-AQ 3.0×50 mm, 5 um; Solvent A=0.1% TFA (trifluoroacetic acid) in water/MeOH (90/10); Solvent B=0.1% TFA in MeOH/water (90/10); B % from 0 to 100% over 5 minutes at flow rate=2.0 ml/min, RT=2.358 minutes. ESI-MS: m/z (M+H)⁺=376.

### 5.4. Synthesis N-(3-(cyclopentyloxy)-4-methoxybenzyl)-6′-morpholino-3,3′-bipyridin-5-amine

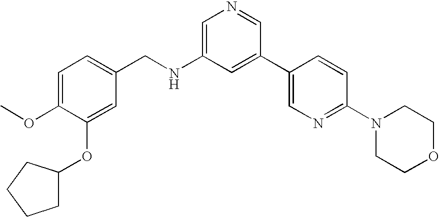

A microwave vial (2 mL) was charged with 5-bromo-N-(3-(cyclopentyloxy)-4-methoxybenzyl)pyridin-3-amine (38 mg, 0.1 mmol) and 6-morpholinopyridin-3-ylboronic acid (20 mg, 0.1 mmol). Then acetonitrile (1 mL), water (0.8 mL), aqueous sodium carbonate (0.2 mL, 1M) and dichlorobis(triphenylphosphine)-palladium(II) (5 mg, 0.007 mmol) were added into the mixture. The reaction vessel was sealed and heated at 150° C. for 5 minutes under microwave irradiation. After cooling, the reaction mixture was worked up and purified by preparative HPLC to give 6 mg of N-(3-(cyclopentyloxy)-4-methoxybenzyl)-6′-morpholino-3,3′-bipyridin-5-amine.

¹H NMR (300 MHz, CD₃OD) δ (ppm): 8.43 (s, 1H), 8.26 (s, 1H), 8.13 (d, J=7.91 Hz, 1H), 7.97 (s, 1H), 7.84 (s, 1H), 7.24 (d, 1H), 6.96 (m, 3H), 4.82 (m, 1H), 4.45 (s, 2H), 3.85 (m, 4H), 3.68 (m, 4H), 3.31 (s, 3H), 1.81 (m, 6H), 1.63 (s, 2H). HPLC: column=YMC Pack ODS-AQ 3.0×50 mm, 5 um; Solvent A=0.1% TFA (trifluoroacetic acid) in water/MeOH (90/10); Solvent B=0.1% TFA in MeOH/water (90/10); B % from 0 to 100% over 5 minutes at flow rate=2.0 ml/min, RT=2.568 minutes. ESI-MS: m/z (M+H)⁺=461.

### 5.5. Synthesis N-(3,4-diisopropoxybenzyl)-5(1H-pyrrol-3-yl)pyridin-3-amine

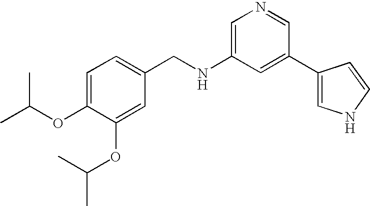

Acetic acid (360 mg, 6 mmol) was added to a solution of 3,4-diisopropoxybenzaldehyde (444 mg, 2 mmol), 5-bromopyridin-3-amine (346 mg, 2 mmol) and sodium triacetoxyborohydride (0.84 g, 4 mmol) in 30 ml DCE at room temperature. The formed mixture was warmed up to 60° C. and stirred for 4 hours. The reaction mixture was quenched with water. The product was extracted with DCM (3×20 ml). The organic layer was separated and dried over sodium sulfate. The organic solvent was evaporated to dryness. The crude product was purified by SiO₂ column chromatography to give 250 mg of 5-bromo-N-(3,4-diisopropoxybenzyl)pyridin-3-amine. Yield: 34%.

Microwave vial (2 mL) was charged with 5-bromo-N-(3,4-diisopropoxybenzyl)-pyridin-3-amine (38 mg, 0.1 mmol) and 1H-pyrrol-3-ylboronic acid (11 mg, 0.1 mmol). Then, acetonitrile (1 mL), water (0.8 mL), aqueous sodium carbonate (0.2 mL, 1M) and dichlorobis(triphenylphosphine)-palladium(II) (5 mg, 0.007 mmol) were added into the mixture. The reaction vessel was sealed and heated at 150° C. for 5 minutes under microwave irradiation. After cooling, the reaction mixture was purified by preparative HPLC to give 6 mg of N-(3,4-diisopropoxybenzyl)-5(1H-pyrrol-3-yl)pyridin-3-amine.

¹H NMR (300 MHz, CD₃OD) δ (ppm): 7.96 (s, 1H), 7.68 (s, 1H), 7.13 (s, 1H), 7.09 (s, 1H), 7.02 (s, 1H), 6.95 (s, 1H), 6.78 (s, 1H), 6.38 (s, 1H), 4.52 (m, 2H), 4.31 (s, 2H), 1.31 (t, 12H). HPLC: column=YMC Pack ODS-AQ 4.6×33 mm, 5 um; Solvent A=0.1% TFA (trifluoroacetic acid) in water/MeOH (90/10); Solvent B=0.1% TFA in MeOH/water (90/10); B % from 0 to 100% over 5 minutes at flow rate=3.0 ml/min, RT=2.826 minutes. ESI-MS: m/z (M+H)⁺=366.

### 5.6. Synthesis N-(3,4-diisopropoxybenzyl)-5-(furan-3-yl)pyridin-3-amine

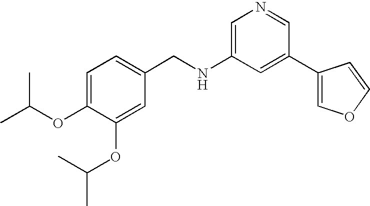

A microwave vial (2 mL) was charged with 5-bromo-N-(3,4-diisopropoxybenzyl)pyridin-3-amine (38 mg, 0.1 mmol) and 1H-pyrrol-3-ylboronic acid (11 mg, 0.1 mmol). Then, acetonitrile (1 mL), water (0.8 mL), aqueous sodium carbonate (0.2 mL, 1M) and dichlorobis(triphenylphosphine)-palladium(II) (5 mg, 0.007 mmol) were added to the mixture. The reaction vessel was sealed and heated at 150° C. for 5 minutes under microwave irradiation. After cooling, the reaction mixture was purified by preparative HPLC to give 5 mg of N-(3,4-diisopropoxybenzyl)-5-(furan-3-yl)pyridin-3-amine.

¹H NMR (300 MHz, CD₃OD) δ (ppm): 7.85 (s, 1H), 7.79 (s, 1H), 7.70 (s, 1H), 7.46 (s, 1H), 7.02 (s, 1H), 6.90 (s, 1H), 6.83 (s, 2H), 6.63 (s, 1H), 4.40 (m, 2H), 4.21 (s, 2H), 1.16 (t, 12H). HPLC: column=YMC Pack ODS-AQ 3.0×50 mm, 5 um; Solvent A=0.1% TFA (trifluoroacetic acid) in water/MeOH (90/10); Solvent B=0.1% TFA in MeOH/water (90/10); B % from 0 to 100% over 5 minutes at flow rate=2.0 ml/min, RT=3.02 minutes. ESI-MS: m/z (M+H)⁺=367.

### 5.7. Synthesis N-(3-(cyclopentyloxy)-4-methoxynemzyl)-6-(furan-3-yl)pyrazin-2-amine

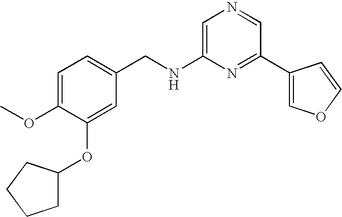

Acetic acid (600 mg, 10 mmol) was added to a solution of 3-(cyclopentyloxy)-4-methoxybenzaldehyde (440 mg, 2 mmol), 6-chloropyrazin-2-amine (258 mg, 2 mmol) and sodium triacetoxyborohydride (1.2 g, 5.6 mmol) in 30 mL dichloroethane at room temperature. The resulting mixture was warmed up to 60° C. and stirred for 4 hours. The reaction mixture was quenched with water. The product was extracted with DCM (3×20 ml). The organic layer was separated and dried over sodium sulfate. The organic solvent was evaporated to dryness. The crude product was purified by SiO₂ column chromatography to give 100 mg of 6-chloro-N-(3-(cyclopentyloxy)-4-methoxybenzyl)pyrazin-2-amine. Yield: 15%

A microwave vial (2 mL) was charged with 6-chloro-N-(3-(cyclopentyloxy)-4-methoxybenzyl)pyrazin-2-amine (40 mg, 0.1 mmol), furan-3-ylboronic acid (11 mg, 0.1 mmol), acetonitrile (1 mL), water (0.8 mL) and aqueous sodium carbonate (0.2 mL, 1M). Then, dichlorobis(triphenylphosphine)-palladium(II) (5 mg, 0.007 mmol) was added into the mixture. The reaction vessel was sealed and heated at 150° C. for 5 minutes under microwave irradiation. After cooling, the reaction mixture was worked up and purified by preparative HPLC to give 1.9 mg of N-(3-(cyclopentyloxy)-4-methoxynemzyl)-6-(furan-3-yl)pyrazin-2-amine.

¹H NMR (300 MHz, CD₃OD) δ (ppm): 7.96 (m, 3H), 7.80 (d, J=8.06 Hz, 1H), 7.74 (t, J=7.91 Hz, 1H), 7.63 (t, J=8.06 Hz, 1H), 7.41 (d, J=8.3 Hz, 2H), 7.21 (m, 1H), 6.69 (s, 1H), 3.87 (m, 1H), 3.34 (m, 1H), 1.17 (t, 1H). HPLC: column=YMC Pack ODS-AQ 3.0×50 mm, 5 um; Solvent A=0.1% TFA (trifluoroacetic acid) in water/MeOH (90/10); Solvent B=0.1% TFA in MeOH/water (90/10); B % from 0 to 100% over 5 minutes at flow rate=3.0 ml/min, RT=3.635 minutes. ESI-MS: m/z (M+H)⁺=366.

### 5.8. Synthesis of N-((9-ethyl-9H-carbazol-3-yl)methyl)-5-(2-methylbenzo [d]thiazol-5-yl)pyrazin-2-amine

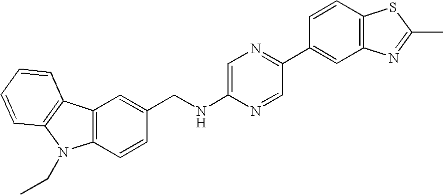

To a solution of (5-bromo-pyrazine-2-yl)-(9-ethyl-9H-carazol-3-ylmethyl)-amine (50 mg, 0.13 mmol) in acetonitrile/water (3:1) solution (2.5 mL) was added 2-methyl-5-(4,4,5,5-tetramethyl-[1,3,2]dioxaborolan-2-yl)-benzothiazol (36 mg, 0.13 mmol), dichlorobis-(triphenylphosphine)palladium(II) (5 mg, 0.007 mmol) and sodium carbonate (28 mg, 0.26 mmol). The resulting mixture was heated under microwave irradiation at 150° C. for 5 minutes. Reaction mixture was diluted with ethyl acetate (10 mL), washed with water, brine, dried and concentrated to give crude product, which was purified by preparative HPLC (10-95% MeOH with 0.1% NH₄OAc) to give desired product (11 mg, 19%).

¹H NMR (400 MHz, MeOD) δ ppm 1.41 (t, J=7.20 Hz, 3H) 2.86 (s, 3H) 4.45 (q, J=7.33 Hz, 2H) 4.79 (s, 2H) 7.16-7.22 (m, J=8.08, 7.07, 1.26 Hz, 1H) 7.42-7.47 (m, J=8.08, 7.07, 1.26 Hz, 1H) 7.50 (d, J=8.34 Hz, 2H) 7.54 (dd, J=8.34, 1.52 Hz, 1H) 7.93 (dd, J=8.59, 1.77 Hz, 1H) 7.97 (t, J=8.08 Hz, 1H) 8.10 (dd, J=4.55, 3.28 Hz, 2H) 8.15 (s, 1H) 8.39 (d, J=1.26 Hz, 1H) 8.57 (d, J=1.26 Hz, 1H). ESI-MS; m/z (M+H)⁺=450.0.

### 5.9. Synthesis of N-(3-(5-(3-(cyclopentyloxy)-4-methoxybenzylamino)pyridin-3-yl)phenyl)methanesulfonamide

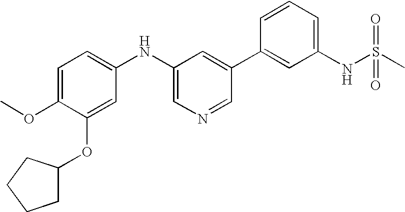

To a microwave vial 5-bromopyridin-3-amine (1.0 g, 5.78 mmol), 3-(methylsulfonamido) phenylboronic acid (1.49 g, 6.94 mmol), CH₃CN (10 mL), CsF (1.69 g, 11.56 mmol), Pd(dppf)Cl₂ (0.85 g, 1.16 mmol) were added and the mixture was heated at 180° C. for 15 minutes. Mixture was cooled to room temperature, concentrated and separated by flash silica gel column chromatography using 1-5% dichloromethane in methanol as solvent to afford N-(3-(5-aminopyridin-3-yl)phenyl)methanesulfonamide (1.14 g, 76% yield).

3-(Cyclopentyloxy)-4-methoxybenzaldehyde (0.046 g, 0.212 mmol), N-(3-(5-amino pyridin-3-yl)phenyl)methanesulfonamide (0.056 g, 0.212 mmol), acetic acid (0.025 g, 0.42 mmol), dichloroethane (5 mL), NaBH(OAc)₃ (0.089 g, 0.42 mmol) were taken in a 10 mL round bottom flask and stirred at 25° C. for 6 h. After the completion of reaction, the mixture was concentrated and separated by preparative HPLC to give 5 mg of N-(3-(5-(3-(cyclopentyloxy)-4-methoxybenzylamino)pyridin-3-yl)phenyl).

¹H NMR (300 MHz, CDCl₃) δ (ppm): 8.10 (m, 2H), 7.30 (m, 5H), 6.80 (m, 3H), 4.70 (m, 1H), 4.30 (s, 2H), 3.76 (s, 3H), 2.97 (s, 3H), 1.76 (m, 5H), 1.52 (m, 3H)HPLC: column=YMC Pack ODS-AQ 4.6×50 mm, 5 um; Solvent A=0.1% TFA (trifluoroacetic acid) in 10% methanol-90% water; Solvent B=0.1% TFA in 90% methanol-10% water. B % from 0 to 100% over 4 minutes at flow rate=3 ml/min, RT=2.560 minutes. ESI-MS: m/z (M+H)⁺=468.

### 5.10. Synthesis of N-{3-[5-(3-Cyclopentyloxy-4-methoxybenzylamino)-pyridin-3-yl]-benzyl}-methanesulfonamide

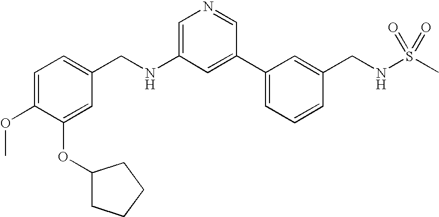

To a 50 mL round bottom flask under nitrogen were added 5-bromopyridin-3-amine (346 mg, 2 mmol) and 3-cyclopentyloxy-4-methoxybenzaldehyde (440 mg, 2 mmol) in 20 ml of dichloroethane. The solution was stirred at room temperature for 10 minutes, then acetic acid (240 mg, 228 ul, 4 mmole) and sodium triacetoxyborohydride (424 mg, 2 mmol) were added. The resulting solution was stirred at room temperature overnight. After the reaction was over, the solution was quenched with water; neutralized with 1 N sodium hydroxide and extracted with methylene chloride. The organic layer was dried over magnesium sulfate and then concentrated in vacuo. The crude product was purified by ISCO SiO₂ chromatography using hexanes/ethyl acetate to give 320 mg of pure compound. Yield: 43%

To a 5 mL microwave reaction vessel were added a solution of 5-bromo-N-(3-cyclopentyloxy)-4-methoxybenzyl)pyridin-3-amine (50 mg, 0.132 mmol), 3-aminomethylphenyl)boronic acid hydrochloride (28 mg, 0.146 mmol, 1.1 equiv.), PdCl₂(PPh₃)₂ (3 mg, 4.27 μmoles, 0.032 equiv.) and sodium carbonate (42 mg, 0.398 mmol, 3 equiv.) in acetonitrile/water (4 mL). The vessel was sealed and the mixture was heated at 155° C. for 5 minutes under microwave irradiation. The mixture was then extracted with water/methylene chloride, the organic layer was dried over magnesium sulfate and filtered through celite. Then removal of solvent gave 42 mg of crude product which was used in next step without further purification. Yield: 79%

[5-(3-Aminomethyl)phenyl)-N-(3-cyclopentyloxy)-4-methoxybenzyl)pyridine-3-amine (20 mg, 49.7 μmoles) was dissolved in 10 ml of dichloromethane. Methanesulfonyl chloride (6.8 mg, 59.7 μmoles, 1.2 equiv.) and pyridine (10 μl 99.4 μmoles, 2 equiv.) were added. The reaction mixture was stirred at 50° C. overnight. Then the reaction mixture was diluted with methylene chloride, washed with water. The organic layer was separated and dried over magnesium sulfate and concentrated under vacuum. The crude product was purified by preparative HPLC to give 4.2 mg of product. Yield: 17%.

¹H NMR (400 MHz, CD₃OD) δ (ppm): 8.21 (s, 1H); 7.94 (s, 1H); 7.84 (s, 1H); 7.68 (s, 1H); 7.59 (m, 1H); 7.54 (m, 2H); 6.98 (m, 3H); 4.80 (m, 1H); 4.22 (s, 2H); 4.18 (s, 2H); 3.81 (s, 3H); 2.93 (s, 3H); 1.80 (m, 6H); 1.61 (m, 2H). HPLC: column=YMC Pack ODS-3×50 mm, 5 um; Solvent A=0.1% TFA (trifluoroacetic acid) in water; Solvent B=0.1% TFA in MeOH/water (95/5); B % from 0 to 100% over 4 minutes at flow rate=2 ml/min, RT=3.21 minutes. ESI-MS: m/z (M+H)⁺=482.

### 5.11. Synthesis of (3-cyclopentyloxy)-4-methoxybenzyl)-[5-(3-methylsulfonyl)phenyl))pyridin-3-amine

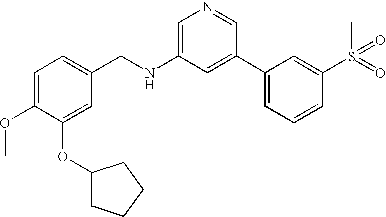

To a 5 mL microwave reaction vessel were added (5-bromo-N-(3-cyclopentyloxy)-4-methoxybenzyl)pyridin-3-amine (50 mg, 0.13 mmole), 3-methylsulfonylphenylboronic acid (27 mg, 0.13 mmol, 1 equiv.), PdCl₂(PPh₃)₂ (4 mg, 0.006 mmol, 0.044 equiv.), sodium carbonate (28 mg, 0.36 mmol, 2 equiv.) and acetonitrile/water 1:1 (4 mL). The sealed vessel was heated at 145° C. for 5 minutes under microwave irradiation. The reaction mixture was then diluted with methylene chloride, washed with water. The organic layer was separated and dried over magnesium sulfate and filtered through Celite. Removal of solvent gave crude product which was purified by preparative HPLC to give 8.4 mg of product. Yield: 13%.

¹H NMR (400 MHz, CD₃OD) δ (ppm): 8.21 (s, 1H), 7.94 (s, 1H), 7.84 (s, 1H), 7.68 (s, 1H), 7.59 (m, 1H), 7.54 (m, 2H), 6.98 (m, 3H), 4.80 (m, 1H) 4.22 (s, 2H), 3.81 (s, 3H), 2.93 (s, 3H), 1.80 (m, 6H), 1.61 (m, 2H). HPLC: column=YMC Pack ODS-3×50 mm, 5 um; Solvent A=0.1% TFA (trifluoroacetic acid) in water; Solvent B=0.1% TFA in MeOH/water (95/5); B % from 0 to 100% over 4 minutes at flow rate=2 ml/min, RT=2.751 min. ESI-MS: m/z (M+H)⁺=453.

### 5.12. Synthesis of N-(3 Cyclopentyloxy)-4-methoxybenzyl)-(5-furan-3-yl)pyridin-3-amine

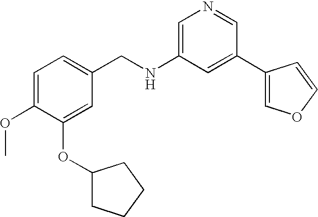

To a 5 mL microwave reaction vessel were added a solution of 5-bromo-N-(3-(cyclopentyloxy)-4-methoxybenzyl)pyridin-3-amine (50 mg, 0.132 mmol), furan-3-yllboronic acid (18 mg, 0.159 mmol, 1.2 equiv.), PdCl₂(PPh₃)₂ (4 mg, 0.006 mmol, 0.044 equiv), sodium carbonate (28 mg, 0.265 mmol, 2 equiv.) and acetonitrile/water 1:1 (4 mL). The vial was heated at 155° C. for 7 minutes under microwave irradiation. The mixture was then extracted with methylene chloride, washed with water. The organic layer was dried over magnesium sulfate and filtered through Celite. Removal of solvent gave the crude product which was purified by preparative HPLC to give 14.1 mg of N-(3 Cyclopentyloxy)-4-methoxybenzyl)-(5-furan-3-yl)pyridin-3-amine. Yield: 29%

¹H NMR (400 MHz, CDCl₃) δ (ppm): 8.19 (s, 1H); 8.11 (s, 1H); 7.81 (s, 1H); 7.55 (s, 1H); 7.35 (s, 1H); 7.28 (s, 1H); 6.88 (m, 2H); 6.61 (s, 1H); 4.80 (m, 1H) 4.38 (s, 2H); 3.85 (s, 3H); 1.88 (m, 6H); 1.61 (m, 2H). HPLC: column=YMC Pack ODS-3×50 mm, 5 um; Solvent A=0.1% TFA (trifluoroacetic acid) in water; Solvent B=0.1% TFA in MeOH/water (95/5); B % from 0 to 100% over 4 minutes at flow rate=2 ml/min, RT=3.55 minutes. ESI-MS: m/z (M+H)⁺=365.

### 5.13. Synthesis of N-(3 Cyclopentyloxy)4-methoxybenzyl)-(1H-pyrrol)pyridin-3-amine

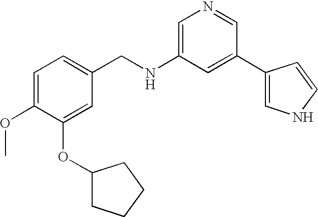

To a 5 mL microwave reaction vessel were added 5-bromo-N-(3-(cyclopentyloxy)-4-methoxybenzyl)pyridin-3-amine (50 mg, 0.13 mmol), 1-(triisopropylsilyl)1H-pyrrol-3-ylboronic acid (49.5 mg, 0.19 mmol, 1.5 equiv.), PdCl₂(PPh₃)₂ (4 mg, 0.006 mmol, 0.044 equiv.), sodium carbonate (28 mg, 0.26 mmol, 2 equiv.) and acetonitrile/water=1/1 (4 mL). The sealed vessel was heated at 155° C. for 7 minutes under microwave irradiation. The mixture was then diluted with methylene chloride and washed with water. The organic layer was then separated and dried over magnesium sulfate and filtered through Celite. Removal of the solvent gave crude product which was purified by preparative HPLC to give 8.02 mg of desired product. Yield: 17%.

¹H NMR (400 MHz, CD₃OD) δ (ppm): 8.05 (s, 1H.); 7.65 (s, 1H); 7.55 (s, 1H); 7.24 (s, 1H); 6.98 (s, 1H); 6.95 (m, 2H); 6.82 (m, 1H); 6.45 (s, 1H); 4.80 (m, 1H) 4.39 (s, 2H); 3.81 (s, 3H); 1.80 (m, 6H); 1.61 (m, 2H). HPLC: column=YMC Pack ODS-3×50 mm, 5 um; Solvent A=0.1% TFA (trifluoroacetic acid) in water; Solvent B=0.1% TFA in MeOH/water (95/5); B % from 0 to 100% over 4 minutes at flow rate=2 ml/min, RT=2.860 minutes. ESI-MS: m/z (M+H)⁺=364.

### 5.14. Synthesis of N-(3 Cyclopentyloxy)-4-methoxybenzyl)-5-(1-(tosyl-1H-indol-3-yl)pyridin-3-amine

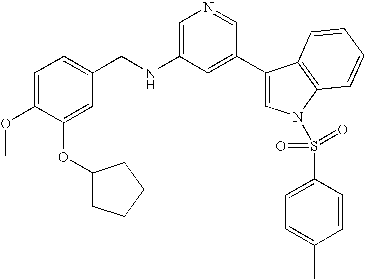

To a 5 ml microwave reaction vessel were added 5-bromo-N-(3-(cyclopentyloxy)4-methoxybenzyl)pyridine-3-amine (50 mg, 0.13 mmol), 1-tosyl-1H-indol-3-boronic acid (54 mg, 0.172 mmole, 1.3 equiv.), PdCl₂(PPh₃)₂ (4 mg, 0.006 mmol, 0.044 equiv.), sodium carbonate (28 mg, 0.26 mmol, 2 equiv.) and acetonitrile/water=1/1 (4 ml). The sealed vessel was heated at 155° C. for 7 minutes under microwave irradiation. The solution was then diluted with methylene chloride and washed with water. The organic layer was separated, dried over magnesium sulfate and filtered through Celite. Removal of the solvent gave crude product which was purified by preparative HPLC to give 9.12 mg of desired product. Yield: 12%

¹H NMR (400 MHz, CD₃OD) δ (ppm): 8.18 (s, 1H.); 8.10(s, 1H); 8.05 (d, 2H); 7.89 (d, 2H); 7.65 (m, 2H); 7.40 (s, 1H); 7.35 (m, 1H); 7.15 (m, 2H); 6.95 (m, 3H); 4.75 (m, 1H) 4.44 (s, 2H); 3.81 (s, 3H); 2.36 (s, 3H) 1.75 (m, 6H); 1.53 (m, 2H). HPLC: column=YMC Pack ODS-3×50 mm, 5 um; Solvent A=0.1% TFA (trifluoroacetic acid) in water; Solvent B=0.1% TFA in MeOH/water (95/5); B % from 0 to 100% over 4 minutes at flow rate=2 ml/min, RT=3.818 minutes. ESI-MS: m/z (M+H)⁺=568.

### 5.15. Synthesis of 3-(3-Cyclopentyloxy-4-methoxy-benzylamino)-5-(3-methanesulfonyl-phenyl)-pyridin-1-ol

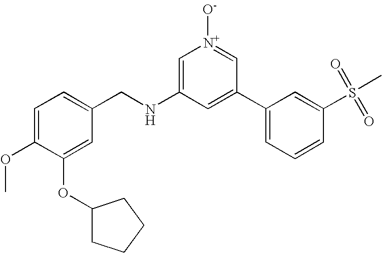

To a solution of 5-bromo-N-(3-cyclopentyloxy)-4-methoxybenzyl)pyridin-3-amine (100 mg, 0.265 mmol) in 10 ml of chloroform was added mCPBA (150 mg, 0.53 mmol, 2 equiv.). The solution was stirred at room temperature overnight. After completion of the reaction, the mixture was quenched with water, washed with saturated sodium bicarbonate aqueous solution and then dried over magnesium sulfate. Removal of the solvent gave 101 mg of product which was used in the next step without further purification. Yield: 97%

To a 5 ml microwave reaction vessel were added 3-bromo-5-(3-cyclopentyloxy-4methoxy-benzylamino)-pyridin-1-ol (50 mg, 0.127 mmol), 3-(methylsulfonyl)phenylboronic acid (28 mg, 1.40 mmol, 1.1 equiv.), PdCl₂(PPh₃)₂ (4 mg, 0.006 mmol), sodium carbonate (28 mg, 0.26 mmol) and acetonitrile/water=1/1 microwave vial (4 mL). The vial was heated at 145° C. for 5 minutes. The solution was then diluted with methylene chloride, washed with water. The organic layer was separated and dried over magnesium sulfate and filtered through Celite. Removal of the solvent gave crude product which was purified by preparative HPLC to give 8.12 mg of desired product, Yield: 13.7%

¹H NMR (400 MHz, CD₃OD) δ (ppm): 8.11 (s, 1H), 8.05 (d, 1H), 7.99 (s, 1H), 7.95 (d, 1H), 7.769 (m, 2H), 7.62 (m, 1H), 7.55 (m, 1H), 6.95 (m, 2H), 4.79 (m, 1H), 4.38 (s, 2H), 3.81 (s, 3H), 3.19 (s, 3H), 1.80 (m, 6H), 1.61 (m, 2H). HPLC: column=YMC Pack ODS-3×50 mm, 5 um; Solvent A=0.1% TFA (Trifluoroacetic acid) in water; Solvent B=0.1% TFA in MeOH/water (95/5); B % from 0 to 100% over 4 minutes at flow rate=2 ml/min, RT=2.978 minutes. ESI-MS: m/z (M+H)⁺=469.

### 5.16. Synthesis 5-(3-methylsulfonyl)phenyl-N-naphthalen-2-ylmethy)pyridin-3-amine

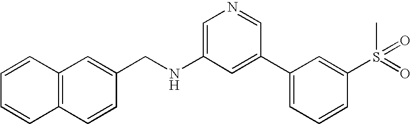

5-Bromopyridin-3-amine (346 mg, 2 mmol, 1 equiv.) was mixed with naphthaldehyde (312 mg, 2 mmol, 1 equiv.) in 20 mL DCE for 10 minutes, acetic acid (240 μL, 4 mmol, 2 equiv.) and sodium triacetoxyborohydride (422 mg, 2 mmol, 1 equiv) were added and the solution was stirred at room temperature overnight. The mixture was then quenched with water, extracted with methylene chloride. The organic layer was separated and dried over magnesium sulfate. Removal of solvent gave crude product which was purified by ISCO SiO₂ column chromatography using hexanes/ethyl acetate to give 320 mg of desired product. Yield: 51%.

To a 5 mL microwave reaction vessel were added (5-bromo-N-(naphtalen-2-ylmethyl)pyridin-3-amine (50 mg, 0.16 mmol), 3-methylsulfonylphenylboronic acid (32 mg, 0.16 mmol), PdCl₂(PPh₃)₂ (4 mg, 0.006 mmol), sodium carbonate (34 mg, 0.32 mmol.) and acetonitrile/water=1:1 (4 mL). The vial was heated at 150° C. for 5 minutes under microwave irradiation. The solution was then diluted with methylene chloride, and washed with water. The organic layer was separated and dried over magnesium sulfate and filtered through Celite. Removal of solvent gave crude product which was purified by preparative HPLC to give 8.4 mg of product Yield: 11%

¹H NMR (400 MHz, CD₃OD) δ (ppm): 8.18 (d, 1H); 7.95 (m, 3H); 7.79 (m, 2H); 7.61 (m, 4H); 7.45 (d, 1H); 7.38 (m, 3H); 4.60 (s, 2H); 3.10 (s, 3H). HPLC: column=YMC Pack ODS-3×50 mm, 5 um; Solvent A=0.1% TFA (trifluoroacetic acid) in water; Solvent B=0.1% TFA in MeOH/water (95/5); B % from 0 to 100% over 4 minutes at flow rate=2 ml/min, RT=3.87 minutes. ESI-MS: m/z (M+H)⁺=469.

### 5.17. Synthesis of N-(biphenyl-2-ylmethyl)-5-(1H-pyrazol-4-yl)pyrazin-2-amine

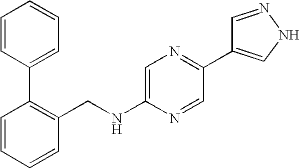

Biphenyl-2-carboxaldehyde (2.0 g, 10.98 mmol) and 5-bromopyrazin-2-amine (1.59 g, 9.15 mmol) were dissolved in acetic acid (2.0 mL) and DCE (5.0 mL). Sodium triacetoxyborohydride (2.91 g, 13.72 mmol) was added and the mixture was stirred at room temperature for 18 hours. The mixture was diluted with CH₂Cl₂, washed with 1.0 N NaOH and brine respectively. Then the organic layer was separated and dried over MgSO₄ and concentrated. The crude material was purified by SiO₂ column chromatography to give 1.5 g of N-(biphenyl-2-ylmethyl)-5-bromopyrazin-2-amine. Yield: 48%

N-(biphenyl-2-ylmethyl)-5-bromopyrazin-2-amine (50 mg, 0.147 mmol), 4-(4,4,5,5-tetramethyl-1,3,2-dioxaborolan-2-yl)-1-H-pyrazole (34 mg, 0.176 mmol), palladiumtriphenylphosphine dichloride (6 mg, 0.0088 mmol), sodium carbonate (34 mg, 0.323 mol), acetonitrile (1.5 mL) and H₂O (1.5 mL) were charged into a 5 mL microwave vial then heated with stirring in a microwave apparatus at 150° C. for 5 minutes. The mixture was cooled, filtered through a syringe filter, and concentrated. The crude material was purified by preparative HPLC (Solvent A=0.1% TFA (trifluoroacetic acid) in water/MeOH (90/10); Solvent B=0.1% TFA in MeOH/water (90/10); B % from 0 to 100% over 12 min; Sunfire 30×50 mm; UV:220) to give 1.5 mg of the title compound, N-(biphenyl-2-ylmethyl)-5-(1-H-pyrazol-4-yl)pyrazin-2-amine. Yield: 3.1%

¹H NMR (400 MHz, CD₃OD) δ (ppm): 8.2 (s, 1H), 8.01 (s, 2H), 7.86 (s, 1H), 7.5 (m, 1H), 7.39 (m, 7H), 7.28 (m, 1H), 4.47 (s, 2H). HPLC: column=ShimPack VP ODS-4.6×50 mm, Solvent A=0.1% TFA (trifluoroacetic acid) in water/MeOH (90/10); Solvent B=0.1% TFA in MeOH/water (90/10); B % from 0 to 100% over 2 minutes at flow rate=3.5 ml/min, RT=2.76 minutes. ESI-MS: m/z (M+H)⁺=328.

### 5.18. Synthesis of N-(3-(cyclopentyloxy)-4-methoxybenzyl)-5-(1H-pyrazol-4-yl)pyridin-3-amine

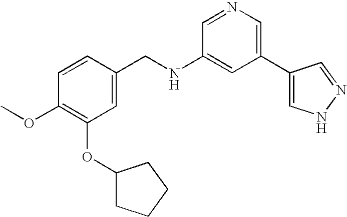

Sodium triacetoxyborohydride (97 mg, 0.46 mmol) was added to a solution of 3-(cyclopentyloxy)-4-methoxybenzaldehyde (50 mg, 0.23 mmol) and 5-bromopyridin-3-amine (39 mg, 0.23 mmol) in 2 mL of 1,2-dichloroethtane (DCE). Acetic acid (18 mg, 0.29 mmol) was added. The mixture was stirred overnight at room temperature, followed by addition of 10 mL of DCE. The organic phase was washed with water, dried over sodium sulfate. Removal of solvent gave 60 mg of crude 5-bromo-N-(3-(cyclopentyloxy)-4-methoxybenzyl)pyridin-3-amine which was used in next step without further purification.

In a 5 ml microwave vial was charged with 5-bromo-N-(3-(cyclopentyloxy)-4-methoxybenzyl)pyridin-3-amine (30 mg, 0.08 mmol), 4-(4,4,5,5-tetramethyl-1,3,2-dioxaborolan-2-yl)-1H-pyrazole (15.4 mg, 0.08 mmol) and acetonitrile (1 mL). Aqueous sodium carbonate (0.16 mL, 1M) and water (0.84 mL) were added to above solution followed by 5 mol % of dichlorobis(triphenylphosphine)-palladium(II) (2.8 mg, 0.004 mmol). The reaction vessel was sealed and heated at 150° C. for 5 minutes under microwave irradiation. After cooling, the reaction mixture was worked up and purified with preparative HPLC to give 3.2 mg of N-(3-(cyclopentyloxy)-4-methoxybenzyl)-5-(1H-pyrazol-4-yl)pyridin-3-amine.

¹H NMR (400 MHz, CD₃OD) δ (ppm): 8.23 (s, 1H), 8.16 (s, 2H), 7.84 (s, 1H), 7.80 (s, 1H), 6.99 (s, 1H), 6.96 (s, 2H), 4.42 (s, 2H), 3.81 (s, 3H), 1.82 (m, 6H), 1.62 (m, 2H). HPLC: YMC Pack ODS-AQ 3.0×50 mm; Solvent A=0.1% TFA (trifluoroacetic acid) in water/MeOH(90/10); Solvent B=0.1% TFA in MeOH/water (90/10); B % from 0 to 100% over 4 minutes at flow rate=2 ml/min, RT=2.57 min. ESI-MS: m/z (M+H)⁺=365.

### 5.19. Synthesis of 2-(4-(5-(3-(cyclopentyloxy)-4-methoxybenzylamino)py-ridin-3-yl)-1H-pyrazol-1-yl)acetamide

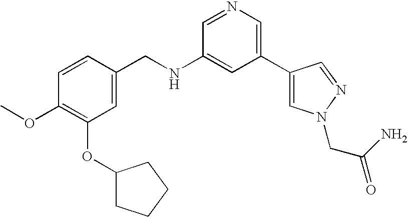

In a 5 ml microwave reaction vial was charged with 5-bromo-N-(3-(cyclopentyloxy)-4-methoxybenzyl)pyridin-3-amine (30 mg, 0.08 mmol), 2-(4-(4,4,5,5-tetramethyl-1,3,2-dioxaborolan-2-yl)-1H-pyrazol-1-yl)acetamide (20 mg, 0.08 mmol), dichlorobis(triphenylphosphine)-palladium(II), (2.8 mg, 0.004 mmol, 5 mol %), acetonitrile (1 mL), aqueous sodium carbonate (0.16 mL, 1M) and water (0.84 mL). The reaction vessel was sealed and heated at 150° C. for 5 minutes under microwave irradiation. After cooling, the reaction mixture was worked up and purified with preparative HPLC to give 6 mg of 2-(4-(5-(3-(cyclopentyloxy)-4-methoxybenzylamino)pyridin-3-yl)-1H-pyrazol-1-yl)acetamide.

¹H NMR (400 MHz, CD₃OD) δ (ppm): 8.23 (s, 1H), 8.19 (s, 1H), 8.00 (s, 1H), 7.79 (s, 2H), 6.97 (s, 1H), 6.95 (s, 2H), 4.94 (s, 2H), 4.84 (m, 1H), 4.41 (s, 2H), 3.80 (s, 3H), 1.82 (m, 6H), 1.62 (m, 2H). HPLC: column=YMC Pack ODS-AQ 3.0×50 mm; Solvent A=0.1% TFA (trifluoroacetic acid) in water/MeOH(90/10); Solvent B=0.1% TFA in MeOH/water (90/10); B % from 0 to 100% over 4 minutes at flow rate=2 ml/min, RT=2.39 minutes. ESI-MS: m/z (M+H)⁺=422.

### 5.20. Synthesis of 5-(furan-3-yl)-N-(1-(naphthalen-2-yl)ethyl)pyridin-3-amine

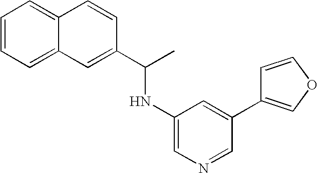

1-(Naphthalene-2-yl)ethanol (200 mg, 1.16 mmol) was dissolved in 5 mL of dichloromethane, triethylamine (351 mg, 3.48 mmol) was added followed by methanesulfonyl chloride (198 mg, 1.74 mmol). The mixture was stirred for 4 hours at room temperature, the formed triethylamine salt was removed by filtration. The filtrate was washed with water and dried over sodium sulfate. Removal of the solvent gave 270 mg of crude 1-(naphthalen-2-yl)ethyl methanesulfonate which was used in next step without further purification.

5-Bromopyridin-3-amine (69 mg, 0.4 mmol) was added to a suspension of sodium hydride (33 mg, 60% in mineral oil, 0.8 mmol) in tetrahydrofuran (4 mL), the mixture was stirred for 30 minutes, then a solution of 1-(naphthalen-2-yl)ethyl methanesulfonate (100 mg, 0.4 mmol) in THF (2 mL) was added. The resulting mixture was heated at 70° C. for 2 hours. After cooling, 2 drops of water were added to quench the reaction. Tetrahydrofuran was evaporated in vacuo. The residue was dissolved in ethyl acetate and washed with water. The organic layer was separated and dried over magnesium sulfate. Removal of solvent gave 100 mg of 5-bromo-N-(1-(naphthalen-2-yl)ethyl)pyridin-3-amine, yield: 73%.

In a microwave reaction vial was charged with 5-bromo-N-(1-(naphthalen-2-yl)ethyl)pyridin-3-amine (20 mg, 0.06 mmol), furan-3-ylboronic acid (14 mg, 0.12 mmol), dichlorobis(triphenylphosphine)-palladium(II) (5 mol %), acetonitrile (1 mL), aqueous sodium carbonate (0.24 mL, 1M) and water (0.76 mL). The reaction vessel was sealed and heated at 150° C. for 5 minutes under microwave irradiation. After cooling, the reaction mixture was evaporated to dryness. The residue was dissolved in 2.5 mL of methanol and purified with preparative HPLC to give 1.6 mg of 5-(furan-3-yl)-N-(1-(naphthalen-2-yl)ethyl)pyridin-3-amine.

¹H NMR (400 MHz, CD₃OD) δ (ppm): 8.07 (s, 1H), 7.90 (s, 2H), 7.88 (s, 1H), 7.84 (s, 1H), 7.82 (s, 1H), 7.76 (m, 1H), 7.65 (m, 2H), 7.56 (s, 1H), 7.47 (m, 2H), 6.80 (s, 1H), 1.70 (d, J=8, 3H). HPLC: column=YMC Pack ODS-AQ 3.0×50 mm; Solvent A=0.1% TFA (trifluoroacetic acid) in water/MeOH(90/10); Solvent B=0.1% TFA in MeOH/water (90/10); B % from 0 to 100% over 4 minutes at flow rate=2 ml/min, RT=3.08 min. ESI-MS: m/z (M+H)⁺=315.

### 5.21. Synthesis of 5-(furan-3-yl)-N-(4-methylbenzyl)pyridin-3-amine

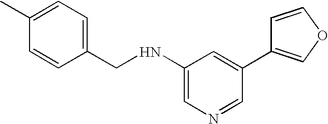

A 20 mL microwave vial was charged with 5-bromopyridin-3-amine (346 mg, 2 mmol), furan-3-ylboronic acid (440 mg, 4 mmol), dichlorobis(triphenylphosphine)-palladium(II) (70 mg, 0.1 mmol), acetonitrile (6 mL), sodium carbonate (6 mL, 1M) and water (0.76 mL). The reaction vessel was sealed and heated at 150° C. for 5 minutes under microwave irradiation. After cooling, the reaction mixture was washed with water and extracted with ethyl acetate; the organic layer was separated and dried over magnesium sulfate. Removal of the solvent gave the crude product which was purified by ISCO SiO₂ column chromatography to give 200 mg of 5-(furan-3-yl)pyridin-3-amine, yield 62%.

Sodium triacetoxyl-borohydride (66 mg, 0.3 μmol) was added to the solution of 5-(furan-3-yl)pyridin-3-amine (25 mg, 0.156 mmol) and 4-methyl-benzaldehyde (19 mg, 0.156 mmol) in 1 mL of 1,2-dichloroethane. Acetic acid (9 mg, 0.156 mmol) was added. The mixture was stirred overnight at room temperature, followed by addition of 5 mL of DCE. The organic phase was washed with water, dried over sodium sulfate. The solvent was removed by rotovap and the residue was purified by preparative HPLC to give 4.4 mg of 5-(furan-3-yl)-N-(4-methylbenzyl)pyridin-3-amine.

¹H NMR (400 MHz, CD₃OD) δ ppm 2.26 (s, 3H) 4.38 (s, 2H) 6.83 (d, J=0.98 Hz, 1H) 7.13 (d, J=7.82 Hz, 2H) 7.24 (d, J=7.82 Hz, 2H) 7.62 (t, J=1.37 Hz, 1H) 7.72 (d, J=1.37 Hz, 1H) 7.78 (d, J=1.95 Hz, 1H) 8.06 (s, 1H), 8.11 (s, 1H). HPLC: column=YMC Pack ODS-AQ 3.0×50 mm; Solvent A=0.1% TFA (trifluoroacetic acid) in water/MeOH(90/10); Solvent B=0.1% TFA in MeOH/water (90/10); B % from 0 to 100% over 4 minutes at flow rate=2 ml/min, RT=2.70 min. ESI-MS: m/z (M+H)³⁰ =265.

### 5.22. Synthesis of 5-(furan-3-yl)-N-(4-isopropoxy-3-methoxybenzyl)pyridine-3-amine

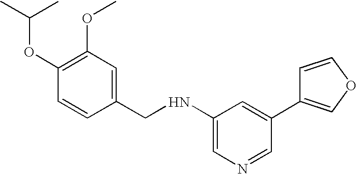

Sodium triacetoxyl-borohydride (66 mg, 0.31 mmol) was added to a solution of 5-(furan-3-yl)pyridin-3-amine (25 mg, 0.156 mmol) and 4-isopropoxy-3-methoxybenzaldehyde (31 mg, 0.156 mmol) in 1 mL of 1,2-dichloroethane. Acetic acid (9 mg, 0.156 mmol) was added. The mixture was stirred overnight at room temperature, followed by addition of 5 mL of DCE. The organic phase was washed with water, dried over sodium sulfate. The solvent was removed by rotovap, and the residue was purified by preparative HPLC to give 11 mg of 5-(furan-3-yl)—N-(4-isopropoxy-3-methoxybenzyl)pyridin-3-amine.

¹H NMR (300 MHz, CD₃OD) δ ppm 1.31(d, J=6 Hz, 6H), 3.84 (s, 3H), 4.43 (s, 2H), 4.53 (m, 1H), 6.92 (d, J=3 Hz, 1H), 6.96 (s, 2H), 7.05 (s, 1H), 7.7 (s, 1H), 7.82 (s, 1H), 7.88 (d, J=3 Hz, 1H), 8.18 (s, 1H), 8.22 (s, 1H). HPLC: YMC Pack ODS-AQ 3.0×50 mm; Solvent A==0.1% TFA (trifluoroacetic acid) in water/MeOH(90/10); Solvent B=0.1% TFA in MeOH/water (90/10); B % from 0 to 100% over 4 minutes at flow rate=2 ml/min, RT=2.68 min. ESI-MS: m/z (M+H)⁺=339.

### 5.23. Synthesis of 1-(4-((1H-imidazol-1-yl)methyl)-4-phenylpiperidin-1-yl)-2,2-diphenylethanone

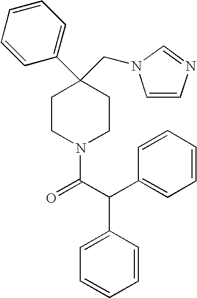

A mixture of 4-(1H-imidazol-1-yl)methyl)-4-phenylpiperidine (80 mg, 0.288 mmol, 1.0 equiv), 2,2-diphenylacetic acid (0.288 mmol, 61 mg, 1 equiv), Polymer bound DCC (234 mg, loading: 1.23 mmol/g, 3 equiv.) and HOBt (0.144 mmol, 19.5 mg, 0.5 equiv.) in THF (10 ml) was stirred at 50° C. for overnight. After completion of the reaction, the polymer reagent was filtered and washed with THF (5 ml). The filtrate was concentrated to give crude product which was purified by preparative HPLC to give 45 mg of 1-(4((1H-imidazol-1-yl)methyl)-4-phenylpiperidin-1-yl)-2,2-diphenylethanone. Yield: 36%

NMR: ¹H-NMR (400 MHz, CD₃OD): δ 1.5 (m, 1H), 1.8 (m, 1H), 2.2 (d, 1H), 2.4 (d, 1H), 2.9 (m, 1H), 3.1 (m, 1H), 4.0 (d, 1H), 4.3 (s, 2H), 4.5 (d, 1H), 5.5 (s, 1H), 7.0 (s, 1H), 7.1-7,5-(m, 16H), 8.1 (s, 1H), Analytical HPLC: RT2.93, (99% purity) M+1: 436(RT: 1.56). ESI-MS: m/z (M+H)⁺=436.

### 5.24. In Vitro Inhibition Assays

Human TPH1, TPH2, tyrosine hydroxylase (TH) and phenylalanine hydroxylase (PH) were all generated using genes having the following accession numbers, respectively: X52836, AY098914, X05290, and U49897.

The full-length coding sequence of human TPH1 was cloned into the bacterial expression vector pET24 (Novagen, Madison, Wis., USA). A single colony of BL21(DE3) cells harboring the expression vector was inoculated into 50 ml of L broth (LB)-kanamycin media and grown up at 37° C. overnight with shaking Half of the culture (25 ml) was then transferred into 3 L of media containing 1.5% Yeast extract, 2% Bacto Peptone, 0.1 mM tryptophan, 0.1 mM ferrous ammonium sulfate, and 50 mM phosphate buffer (pH 7.0), and grown to OD₆₀₀=6 at 37° C. with oxygen supplemented at 40%, pH maintained at 7.0, and glucose added. Expression of TPH1 was induced with 15% D-lactose over a period of 10 hours at 25° C. The cells were spun down and washed once with phosphate buffered saline (PBS).

TPH1 was purified by affinity chromatography based on its binding to pterin. The cell pellet was resuspended in a lysis buffer (100 ml/20 g) containing 50 mM Tris-Cl, pH 7.6, 0.5 M NaCl, 0.1% Tween-20, 2 mM EDTA, 5 mM DTT, protease inhibitor mixture (Roche Applied Science, Indianapolis, Ind., USA) and 1 mM phenylmethanesulfonyl fluoride (PMSF), and the cells were lyzed with a microfluidizer. The lysate was centrifuged and the supernatant was loaded onto a pterin-coupled sepharose 4B column that was equilibrated with a buffer containing 50 mM Tris, pH 8.0, 2 M NaCl, 0.1% Tween-20, 0.5 mM EDTA, and 2 mM DTT. The column was washed with 50 ml of this buffer and TPH1 was eluded with a buffer containing 30 mM NaHCO₃, pH 10.5, 0.5 M NaCl, 0.1% Tween-20, 0.5 mM EDTA, 2 mM DTT, and 10% glycerol. Eluted enzyme was immediately neutralized with 200 mM KH₂PO₄, pH 7.0, 0.5 M NaCl, 20 mM DTT, 0.5 mM EDTA, and 10% glycerol, and stored at −80° C.

Human tryptophan hydroxylase type II (TPH2), tyrosine hydroxylase (TH) and phenylalanine hydroxylase (PAH) were expressed and purified essentially in the same way, except the cells were supplemented with tyrosine for TH and phenylalanine for PAH during growth.

TPH1 and TPH2 activities were measured in a reaction mixture containing 50 mM 4-morpholinepropanesulfonic acid (MOPS), pH 7.0, 60 uM tryptophan, 100 mM ammonium sulfate, 100 uM ferrous ammonium sulfate, 0.5 mM Tris(2-carboxyethyl)phosphine (TCEP), 0.3 mM 6-methyl tetrahydropterin, 0.05 mg/ml catalase, and 0.9 mM DTT. The reactions were initiated by adding TPH1 to a final concentration of 7.5 nM. Initial velocity of the reactions was determined by following the change of fluorescence at 360 nm (excitation wavelength=300 nm). TPH1 and TPH2 inhibition was determined by measuring their activities at various compound concentrations, and the potency of a given compound was calculated using the equation:

$$v = b + \frac{{v}_{0} - b}{1 + {( \frac{[ C ]}{[ {I}_{c 50} ]} )}^{D}}$$

Where v is the initial velocity at a given compound concentration C, v₀ is the v when C=0, b is the background signal, D is the Hill slope which is approximately equal to 1, and I_{C50} is the concentration of the compound that inhibits half of the maximum enzyme activity.

Human TH and PAH activities were determined by measuring the amount of ³H₂O generated using L-[3,4-³H]-tyrosine and L-[4-³H]-phenylalanine, respectively. The enzyme (100 nM) was first incubated with its substrate at 0.1 mM for ˜10 minutes, and added to a reaction mixture containing 50 mM MOPS, pH 7.2, 100 mM ammonium sulfate, 0.05% Tween-20, 1.5 mM TCEP, 100 uM ferrous ammonium sulfate, 0.1 mM tyrosine or phenylalanine, 0.2 mM 6-methyl tetrahydropterin, 0.05 mg/ml of catalase, and 2 mM DTT. The reactions were allowed to proceed for 10-15 minutes and stopped by the addition of 2 M HCl. The mixtures were then filtered through activated charcoal and the radioactivity in the filtrate was determined by scintillation counting. Activities of LX1031 on TH and PAH were determined using this assay and calculated in the same way as on TPH1 and TPH2.

### 5.25. Cell-Based Inhibition Assays

Two types of cell lines were used for screening: RBL2H3 is a rat mastocytoma cell line, which contains TPH1 and makes 5-hydroxytrypotamine (5HT) spontaneously; BON is a human carcinoid cell line, which contains TPH1 and makes 5-hydroxytryptophan (5HTP). The CBAs were performed in 96-well plate format. The mobile phase used in HPLC contained 97% of 100 mM sodium acetate, pH 3.5 and 3% acetonitrile. A Waters C18 column (4.6×50 mm) was used with Waters HPLC (model 2795). A multi-channel fluorometer (model 2475) was used to monitor the flow through by setting at 280 nm as the excitation wavelength and 360 nm as the emission wavelength.

RBL CBA: Cells were grown in complete media (containing 5% bovine serum) for 3-4 hours to allow cells to attach to plate wells (7K cell/well). Compounds were then added to each well in the concentration range of 0.016 μM to 11.36 μM. The controls were cells in complete media without any compound present. Cells were harvested after 3 days of incubation at 37° C. Cells were >95% confluent without compound present. Media were removed from plate and cells were lysed with equal volume of 0.1 N NaOH. A large portion of the cell lysate was treated by mixing with equal volume of 1M TCA and then filtered through glass fiber. The filtrates were loaded on reverse phase HPLC for analyzing 5HT concentrations. A small portion of the cell lysate was also taken to measure protein concentration of the cells that reflects the cytotoxicity of the compounds at the concentration used. The protein concentration was measured by using BCA method.

The average of 5HT level in cells without compound treated was used as the maximum value in the IC₅₀ derivation according to the equation provided above. The minimum value of 5HT is either set at 0 or from cells that treated with the highest concentration of compound If a compound is not cytotoxic at that concentration.

BON CBA: Cells were grown in equal volume of DMEM and F12K with 5% bovine serum for 3-4 hours (20K cell/well) and compound was added at a concentration range of 0.07 μM to 50 μM. The cells were incubated at 37° C. overnight. Fifty μM of the culture supernatant was then taken for 5HTP measurement. The supernatant was mixed with equal volume of 1M TCA, then filtered through glass fiber. The filtrate was loaded on reverse phase HPLC for 5HTP concentration measurement. The cell viability was measured by treating the remaining cells with Promega Celltiter-Glo Luminescent Cell Viability Assay. The compound potency was then calculated in the same way as in the RBL CBA.

All of the publications (e.g., patents and patent applications) disclosed above are incorporated herein by reference in their entireties.

1. A method of inhibiting TPH, which comprises contacting TPH with an effective amount of a TPH inhibitor of the formula: 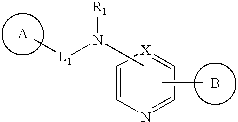
or a pharmaceutically acceptable salt thereof, wherein:
X is C or N;
A is optionally substituted aryl or heteroaryl;
B is optionally substituted aryl or heteroaryl;
L₁ is —(CR₂)ₘ—;
R₁ is hydrogen or optionally substituted alkyl;
each R₂ is independently hydrogen or optionally substituted alkyl; and
m is 0 or 1.

2. The method TPH of claim 1, wherein the TPH inhibitor of the formula: 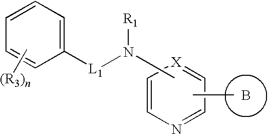
wherein:
each R₃ is independently optionally substituted alkyl, heteroalkyl, aryl, heterocycle, alkylaryl, heteroalkyl-aryl, alkyl-heterocycle, or heteroalkyl-heterocycle; and
n is 0-4.

3. The method of claim 2, wherein R₁ is hydrogen.

4. The method of claim 2, wherein R₂ is hydrogen.

5. The method of claim 2, wherein at least one R₃ is alkoxy.

6. The method of claim 2, wherein m is 0.

7. The method of claim 2, wherein m is 1.

8. The method of claim 2, wherein the TPH inhibitor is of the formula:

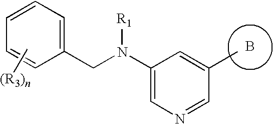

9. The method of claim 8, wherein the TPH inhibitor is of the formula: 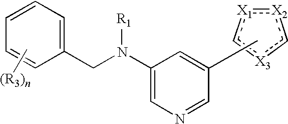
wherein:
X₁ is N, NR₄, O, CHR₅, or CR₅;
X₂ is N, NR₄, O, CHR₅, or CR₅;
X₃ is N, NR₄, O, CHR₅, or CR₅;
each R₄ is independently hydrogen or optionally substituted alkyl, heteroalkyl, aryl, heterocycle, alkylaryl, heteroalkyl-aryl, alkyl-heterocycle, or heteroalkyl-heterocycle; and
each R₅ is independently hydrogen or optionally substituted alkyl, heteroalkyl, aryl, heterocycle, alkylaryl, heteroalkyl-aryl, alkyl-heterocycle, or heteroalkyl-heterocycle.

10. The method of claim 9, wherein X₁ is O and X₂ and X₃ are both CHR₅.

11. The method of claim 10, wherein R₅ is hydrogen.

12. The method of claim 9, wherein X₁ is N, X₂ is NR₄, and X₃ is CHR₅.

13. The method of claim 10, wherein R₄ is optionally substituted alkyl or heteroalkyl, and R₅ is hydrogen or optionally substituted alkyl.

14. The method of claim 8, which is of the formula: 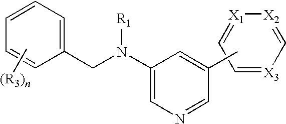
wherein:
X₁ is N or CR₄;
X₂ is N or CR₄;
X₃ is N or CR₄; and
each R₄ is independently hydrogen or optionally substituted alkyl, heteroalkyl, aryl, heterocycle, alkylaryl, heteroalkyl-aryl, alkyl-heterocycle, or heteroalkyl-heterocycle.

15-22. (canceled)
